(19) United States
(12) Patent Application Publication
(10) Pub. No.: US 2008/0135055 A1
(43) Pub. Date: Jun. 12, 2008

(54) Elastic barrier for use with male condom

(76) Inventor: Lance Adam Portnoff, Mill Valley, CA (US)

Correspondence Address: STEVEN A. NIELSEN;ALLMAN & NIELSEN, P.C, 100 Larkspur Landing Circle, Suite 212, LARKSPUR, CA, 94939, US

(21) Appl. No.: 11/608,722

(22) Filed: Dec. 8, 2006

(51) Int. Cl. A61F 6/02

(52) U.S. Cl. 128/842

(57) ABSTRACT

A circular membrane surrounds the penis and provides a flexible barrier over the pubic area. A penis fits through an opening in the membrane and though a short circular shaft. A condom may be fitted on the penis and rolled down the circular shaft, creating an overlapping seal and reducing skin and fluid contact. A raised ridge at the perimeter of the circular membrane prevents the transfer of fluid from the membrane.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND OF THE INVENTION

(1) Field of the Invention

The invention relates to barriers for use during sexual contact.

(2) Description of the Related Art

Several attempts to minimize risk during sexual contact are known in the related art. The use of condoms is well known, but does nothing to prevent contact in the pubic area. Thus, there is room in the art for means of protecting the pubic area during sexual contact.

### BRIEF SUMMARY OF THE INVENTION

The present invention overcomes shortfalls in the related art by combining the advantages of a separate condom and a separate flexible barrier that fits between the penis and a condom. The invention allows a condom to be rolled down a penis and over a separate barrier that protects the pubic area. The invention may be used with any condom suitable to the user.

The invention is designed to prevent any skin-to-skin contact during sexual intercourse.

By using the invention with a condom, disease that is contracted by skin-to-skin contact or with body fluids is greatly reduced or eliminated.

The invention is used by simply sliding the tube or shaft section of the invention down an erect penis, and then rolling down any brand condom over the penis and shaft section of the invention. By utilizing the invention with a condom, a secure barrier is created protecting the wearer from any skin or fluid contact while engaging in sexual activity.

These and other objects and advantages will be made apparent when considering the following detailed specification when taken in conjunction with the drawings.

### BRIEF DESCRIPTION OF THE DRAWINGS

FIG. 1 is a plan view of the bottom of the invention.

FIG. 2 is an elevation view of the side of the invention.

### DETAILED DESCRIPTION OF THE INVENTION

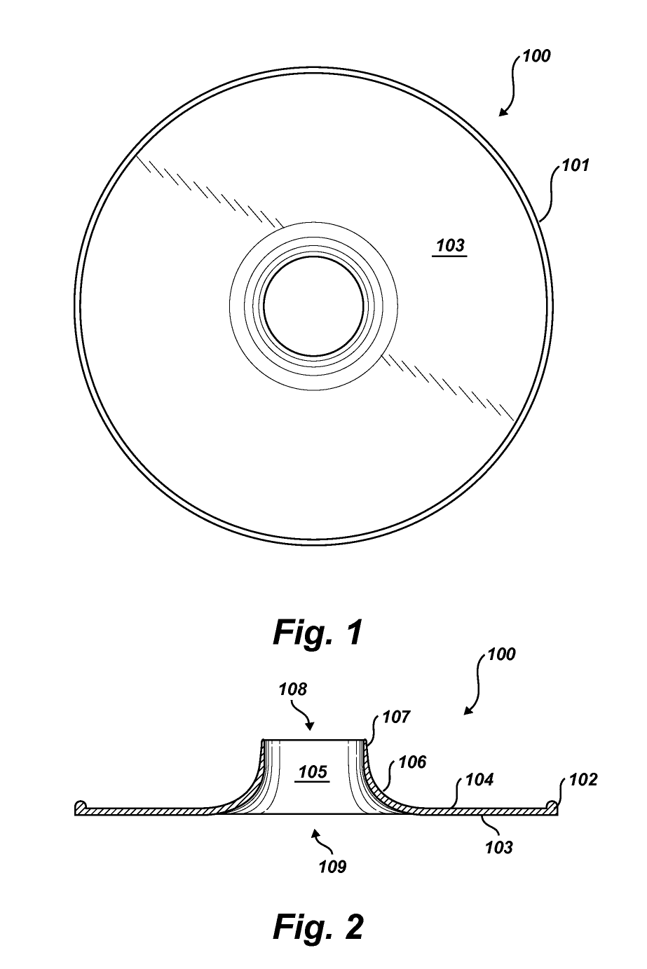

FIG. 1 shows a bottom plan view of the invention 100. FIG. 1 illustrates the bottom of the outer elevated ring 101. FIG. 2 illustrates the raised ridge 102 of the outer elevated ring. The outer elevated ring 102 circles the perimeter of the circular body. The bottom of the circular body 103 covers the pubic area. The top of the circular body 104 connects to the tube section 105. A condom of any brand my roll over the tube section 105.

The tube section 105 has an entering opening 109 at the bottom and an exiting opening 108 at the top. A penis first enters the bottom 109 opening and protrudes through the top opening 108. To facilitate the rolling of a condom over the tube section, the bottom 106 of the tube section is wider than the top 107 of the tube section.

### Preferred Embodiment

In the preferred embodiment, the diameter of the invention 100 is approximately 15 cm or 6 inches and the tube section 105 is approximately 4 cm or 1.5 inches in height. The perimeter ridge section 102 is normal to the circular body section 104. The perimeter ridge section 102 is approximately 0.5 cm or ¼ of an inch in height and is raised in parallel to the tube section 105.

The perimeter ridge section 102 prevents sweat and or other fluids or material from sliding from the circular body 104 section to the user of the invention. The perimeter ridge section 102 also acts as a holding point for applying and removing the invention.

The invention is preferably made of latex but may be made of any flexible material such as polyurethane.

What is claimed is:

1. A flexible, circular, non-permeable shield comprising:
a) an opening at the center of the shield suitable for entry by an erect penis;
b) a shaft section at the top of the shield and surrounding the center opening; and
c) a raised ridge section at the outer perimeter of the shield.

2. The shield of claim 1 comprising a shaft section that is wider at the shield connection as compared to the top portion of the shaft section.

3. The shield of claim 1 that is 6 inches or more in diameter.

4. The shield of claim 1 comprising a shaft section that is 1.5 inches or more in length.

5. The shield of claim 1 comprising a perimeter ridge section of ¼ inches or more in height.

6. The shield of clam 1 made of latex.

7. The shield of claim 1 made of polyurethane.

8. A method of reducing exposure to skin and fluids during sexual activity comprising the use of a flexible, circular, non-permeable shield having:
a) an opening at the center of the shield suitable for entry by an erect penis;
b) a shaft section at the top of the shield and surrounding the center opening; and
c) a raised ridge section at the outer perimeter of the shield.

9. The method of claim 8 comprising the use of the shield with a condom rolled down a penis and over the shaft section of the shield.

10. The method of claim 8 wherein the raised ridge section is used to hold the shield when inserting the shield over a penis.

11. The method of claim 8 wherein the raised ridge section is used to hold the shield when removing the shield from the user.
